SEQUENCE LISTING (28 sequences)

SEQ ID NO: 1 - protein, 106 aa, Homo Sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 227 aa, Homo Sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 345 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 11 aa, Artificial Sequence
  Synthetic Linker
GTELSVRAKPS

SEQ ID NO: 5 - protein, 116 aa, Homo Sapiens
EEELQVIQPDKSVSVAAGESAILHCTVTSLIPVGPIQWFRGAGPARELIYNQKEGHFPRVTTVSTKRENMDFSISISNITPADAGTYYCVKFRKGSPDTEFKSGAGTELSVRAKPS

SEQ ID NO: 6 - protein, 229 aa, Homo Sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 229 aa, Homo Sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 345 aa, Homo Sapiens; identical to SEQ ID NO: 3

SEQ ID NO: 9 - protein, 347 aa, Homo Sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 216 aa, Homo Sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 11 - protein, 5 aa, Artificial Sequence
  Synthetic Linker
GGGGS

SEQ ID NO: 12 - protein, 12 aa, Artificial Sequence
  Synthetic Ligand
  MISC_FEATURE (2)..(2): Xaa is 2,3-bispalmitoyloxypropyl
SXCGDPKHPKSF

SEQ ID NO: 13 - protein, 504 aa, Homo Sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 14 - protein, 503 aa, Homo Sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 15 - protein, 330 aa, Homo Sapiens [sequence omitted]

SEQ ID NO: 16 - protein, 473 aa, Homo Sapiens [sequence omitted]

SEQ ID NO: 17 - protein, 471 aa, Homo Sapiens [sequence omitted]

SEQ ID NO: 18 - protein, 471 aa, Homo Sapiens [sequence omitted]

SEQ ID NO: 19 - protein, 330 aa, Homo Sapiens
ASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 469 aa, Homo Sapiens [sequence omitted]

SEQ ID NO: 21 - protein, 474 aa, Homo Sapiens [sequence omitted]

SEQ ID NO: 22 - protein, 475 aa, Homo Sapiens [sequence omitted]

SEQ ID NO: 23 - protein, 469 aa, Homo Sapiens [sequence omitted]

SEQ ID NO: 24 - protein, 326 aa, Homo Sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 25 - protein, 377 aa, Homo Sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 26 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 327 aa, Homo Sapiens
ASFKGPSVFPLVPCSRSTSESTAALGCLVKDYFPEPVTVSWNSCALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVRVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSPGK

SEQ ID NO: 28 - protein, 344 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2019/0091290 A1
(43) Pub. Date: Mar. 28, 2019

(54) MACROPHAGE STIMULATION IN CD47 BLOCKADE THERAPY

(71) Applicant: TRILLIUM THERAPEUTICS INC., Mississauga (CA)

(72) Inventors: Gloria Hoi Ying Lin, Oakville (CA); Natasja Nielsen Viller, Oakville (CA); Lisa Danae Schultz Johnson, Etobicoke (CA); Mark Michael Wong, Toronto (CA); Robert Adam Uger, Richmond Hill (CA)

(21) Appl. No.: 16/092,168

(22) PCT Filed: Apr. 13, 2017
(86) PCT No.: PCT/CA17/50458

§ 371 Date: Oct. 8, 2018

Related U.S. Application Data
(60) Provisional application No. 62/322,934, filed on Apr. 15, 2016.

(51) Int. Cl. A61K 38/17; A61K 38/21; A61P 35/00

(52) U.S. Cl. CPC A61K 38/177; A61P 35/00; A61K 38/217

(57) ABSTRACT

Blockade of the CD47/SIRPa pathway depletes cancer cells. This anti-cancer activity is enhanced when macrophage stimulating agents are used in combination with the CD47 blockade drug. This anti-cancer combination therapy is particularly effective when the CD47 blockade drug is SIRPaFc.

This application claims the benefit under 35 USC § 119(e) of U.S. Provisional application No. 62/322,934 filed Apr. 15, 2016 which is incorporated herein by reference.

### FIELD OF THE INVENTION

This invention relates to methods of using a drug that blocks the CD47/SIRPa interaction. More particularly, the invention relates to improvements that result when cancer patients receiving a SIRPαFc drug are also treated to stimulate macrophage populations.

### BACKGROUND TO THE INVENTION

Cancer cells are targeted for destruction by antibodies that bind to cancer cell antigens, and through recruitment and activation of macrophages by way of Fc receptor binding to the Fc portion of that antibody. Binding between CD47 on cancer cells and SIRPa on macrophages transmits a “don't eat me” signal that enables many tumour cells to escape detection and destruction by macrophages. It has been suggested that inhibition of the CD47/SIRPa interaction (SIRPαFc) will allow macrophages to “see” and destroy the target CD47+ cancer cell. The use of SIRPα-based agents to treat cancer by SIRPαFc is described in WO2010/130053. (SIRPa and SIRPα are used interchangeably herein as equivalent terms for SIRPalpha. Likewise, SIRPaFc and SIRPαFc are used interchangeably herein.)

In WO2014/094122, we describe a drug that inhibits interaction between CD47 and SIRPa. This SIRPαFc drug is a form of human SIRPa that incorporates a particular region of its extracellular domain linked with a particularly useful form of an IgG1-based Fc region. In this form, the SIRPaFc drug shows dramatic effects on the viability of cancer cells that present with a CD47+ phenotype. The effect is seen particularly on acute myelogenous leukemia (AML) cells, and on many other types of cancer. A soluble form of SIRP having significantly altered primary structure and enhanced CD47 binding affinity is described in Stanford's WO2013/109752. Another similar form of SIRPaFc drug that comprises a tumour antigen binding site is described in Merck GMBH's WO2016/024021.

Other SIRPαFc drugs have been described in the literature and these include various CD47 antibodies (see for instance Stanford's U.S. Pat. No. 8,562,997, and InhibRx′ WO2014/123580), each comprising different antigen binding sites but having, in common, the ability to compete with endogenous SIRPa for binding to CD47, thereby to allow for phagocytosis and, ultimately, an increase in the rate of CD47+ cancer cell depletion. These drugs, while having a SIRPαFc effect, show activities in vivo that are quite different from those displayed by SIRPaFc-based drugs. The latter, for instance, display negligible binding to red blood cells whereas the opposite property in CD47 antibodies creates a need for strategies that accommodate the drug “sink” that follows administration.

Still other agents are proposed for use in blocking the CD47/SIRPa axis. These include CD47Fc proteins (see Viral Logic's WO2010/083253), and SIRPa antibodies as described in UHN's WO2013/056352, Stanford's WO2016/022971, Eberhard's U.S. Pat. No. 6,913,894, and elsewhere.

The mechanism by which these drugs exert their effects is not fully understood. It is believed that a benefit is realized directly from the inhibition of CD47 signalling. However, it is also likely that macrophages cooperate in some way to promote cancer cell depletion.

The CD47 blockade approach shows great promise in cancer therapy. It would be useful to provide methods and means for improving the effect of these drugs, and in particular for improving the effect of the SIRPαFc drugs.

### SUMMARY OF THE INVENTION

It has been determined that the anti-cancer effect of a SIRPαFc drug is enhanced when a recipient is treated in combination with one or more agents that are macrophage stimulating agents. In embodiments, the endogenous macrophages so stimulated are tumour associated macrophages (TAMs).

Thus, in one aspect, there is provided a method useful to deplete CD47+ disease cells in a subject in need thereof, comprising administering to the subject (1) SIRPaFc as a CD47 blockade drug, and (2) a macrophage stimulating agent effective to activate endogenous macrophages, thereby to enhance anti-cancer activity of the SIRPaFc drug.

In certain aspects, the macrophage stimulating agent is one that supports or favours formation or accumulation of macrophages that, in the context of Fc receptor types, are CD64+. In other aspects, the macrophage stimulating agent is one that supports formation of macrophages that have an M1 type or an M2c type. In yet other aspects, the macrophage stimulating agent is one that polarizes M0, M2a and M2b type macrophages to become M1 and/or M2c macrophages.

In a related aspect, there is provided the use of a SIRPαFc drug in combination with a macrophage stimulating agent effective to modulate the activity and/or phenotype of endogenous macrophages, including TAMS, thereby to deplete CD47+ disease cells in a subject in need thereof.

In another aspect, there is provided a pharmaceutical combination useful to deplete CD47+ disease cells in a subject in need thereof, the combination comprising a SIRPαFc drug in combination with a macrophage stimulating agent. In embodiments, the combination is provided as a physical combination of discrete and separately formulated compounds, such as a kit, together with instructions teaching their use in the treatment method herein described.

In embodiments, the macrophage stimulating agent is selected from interferon-gamma (IFNγ), interferon alpha such as interferon-alpha 2a (IFNα-2a) lipopolysaccharide (LPS), and interleukins such as interleukin 1β, interleukin 4, and interleukin 10 (IL-10), colony stimulating factors such as M-CSF and GM-CSF, transforming growth factor beta (TGFβ), toll-like receptor (TLR) ligand, heat aggregated human gamma globulin (HAGG), and mixtures of any two or more thereof

These and other aspects of the invention are now described in greater detail with reference to the accompanying drawings, in which:

### BRIEF REFERENCE TO THE DRAWINGS

FIG. 1 shows SIRPaFc (SEQ ID NO: 3, TTI-621) increased phagocytosis of tumor cells by 6 different macrophage subsets that were generated from human PBMC in vitro;

FIG. 2A shows the relative expression of FcϰRs (CD16, CD32, CD64) in the 6 different macrophage subsets that were generated from human PBMC in vitro. FIG. 2B shows the correlation between CD64 levels on the 6 different macrophage subsets and phagocytic activity by SIRPaFc;

FIGS. 3A-3C show that phagocytic response to SIRPaFc (SEQ ID NO: 3) by M0, M2a and M2b macrophages are further increased by re-polarization with cytokines and Toll-like receptor agonists;

FIG. 4 shows the changes in FcϰR expression of M0, M2a and M2b macrophages after re-polarization with cytokines and Toll-like receptor agonists. Most notable are the changes in CD64 expression; and

FIGS. 5A-5C shows tumor growth in mice treated with SIRPaFc (SEQ ID NO: 3) in combination with interferon gamma.

### DETAILED DESCRIPTION AND PREFERRED EMBODIMENTS

The present invention provides an improved method for treating subjects presenting with cancer cells and tumours that have a CD47+ phenotype. In this method, subjects receive a combination of SIRPaFc and a macrophage stimulating agent. In combination, the anti-cancer effect of this combination is superior to the effects of either agent alone. The improvement is believed to result particularly when the drug is a SIRPaFc drug having SEQ ID NO: 3.

Thus, in the present invention, the treatment method combines a SIRPaFc drug, and a macrophage stimulating agent. A drug that has SIRPαFc activity is an agent that interferes with signal transmission that results when CD47 interacts with macrophage-presented SIRPa. This property is found in various forms of a SIRPaFc drug.

SIRPαFc drugs are based on the extracellular region of human SIRPa. They comprise at least a region of the extracellular region sufficient to confer effective CD47 binding affinity and specificity. Some SIRPaFc drugs are described in the literature and include those referenced in Novartis' WO 2010/070047 and Stanford's WO2013/109752, as well as Trillium Therapeutics' WO 2014/094122, all incorporated herein by reference in their entireties.

In a SIRPaFc drug, a CD47-binding region of SIRPa is coupled to an antibody constant domain (Fc), to form a SIRPaFc fusion. More particularly, the drug suitably comprises an extracellular part of the human SIRPα protein, in a form fused directly, or indirectly, with an antibody constant region, or Fc (fragment crystallisable) Unless otherwise stated, the term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (SEQ ID NO: 13) (residues 27-504 constitute the mature form). Another form, the variant 2 or V2 form, differs by 13 amino acids and has the amino acid sequence set out in GenBank as CAA71403.1 (SEQ ID NO: 14) (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of triggering signal transduction through CD47 upon binding thereto. The present invention is directed most particularly to the drug combinations that include the variant 2 form, or V2.

In the present drug combination, useful SIRPαFc fusion proteins comprise one of the three so-called immunoglobulin (Ig) domains that lie within the extracellular region of human SIRPα. More particularly, the present SIRPαFc proteins incorporate residues 32-137 of human SIRPα (a 106-mer), which constitute and define the IgV domain of the V2 form according to current nomenclature. This SIRPα sequence, shown below, is referenced herein as SEQ ID NO: 1.

(SEQ ID NO: 1)
EELQVIQPDKSVSVAAGESAILHCTVTSLIPVGPIQWFRGAGPARELIYN
QKEGHFPRVTTVSESTKRENMDFSISISNITPADAGTYYCVKFRKGSPDT
EFKSGA

In embodiments, the SIRPαFc fusion proteins incorporate the IgV domain as defined by SEQ ID NO: 1, and additional, flanking residues contiguous within the SIRPα sequence. This form of the IgV domain, represented by residues 31-148 of the V2 form of human SIRPα, is a 118-mer having SEQ ID NO: 5 shown below:

(SEQ ID NO: 5)
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPS

As SIRPαFc drugs, the SIRPα fusion proteins can also incorporate an Fc region having effector function. Fc refers to “fragment crystallisable” and represents the constant region of an antibody comprised principally of the heavy chain constant region and components within the hinge region. Suitable Fc components thus are those having effector function. An Fc component “having effector function” is an Fc component having at least some effector function, such as at least some contribution to antibody-dependent cellular cytotoxicity or some ability to fix complement. Also, the Fc will at least bind to Fc receptors. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Va1234, Ala235 and deletion of Gly236 (EU), is considered not to have effector function. In a preferred embodiment, the Fc is based on human antibodies of the IgG1 isotype. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 (SEQ ID NO: 15) in UniProtKB/Swiss-Prot, residues 104-330, and has the amino acid sequence shown below and referenced herein as SEQ ID NO: 2:

(SEQ ID NO: 2)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK*

Thus, the Fc region has either a wild type or consensus sequence of an IgG1 constant region. Alternatively, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (SEQ ID NO: 16) (residues 242-473), BAC04226.1 (SEQ ID NO: 17) (residues 247-478), BAC05014.1 (SEQ ID NO: 18) (residues 240-471), CAC20454.1 (SEQ ID NO: 19) (residues 99-320), BAC05016.1 (SEQ ID NO: 20) (residues 238-469), BAC85350.1 (SEQ ID NO: 21) (residues 243-474), BAC85529.1 (SEQ ID NO: 22) (residues 244-475), and BAC85429.1 (SEQ ID NO: 23) (residues (238-469).

In the alternative, the Fc region can be a wild type or consensus sequence of an IgG2 or IgG3 sequence, examples thereof being shown below:

a human IgG2, for example:

(SEQ ID NO: 9)
APPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAP
IEKTISKTKGQPREPQVYTLPPSREEMTKNQVSLTCLVKGFYPSDISVEW
ESNGQPENNYKTTPPMLDSDGSFFLYSKLTVDKSRWQQGNVFSCSVMHEA
LHNHYTQKSLSLSPGK,

as comprised in P01859 (SEQ ID NO: 24) of the UniProtKB/Swiss-Prot database;

a human IgG3, for example:

(SEQ ID NO: 10)
APELLGGPSVFLFPPKPKDTLMISRTPEVTCVVVDVSHEDPEVQFKWYVD
GVEVHNAKTKPREEQYNSTFRVVSVLTVLHQDWLNGKEYKCKVSNKALPA
PIEKTISKTKGQPREPQVYTLPPSREEMTKNQVSLTCLVKGFYPSDIAVE
WESSGQPENNYNTTPPMLDSDGSFFLYSKLTVDKSRWQQGNIFSCSVMHE
ALHNRFTQKSLSLSPGK,

as comprised in P01860 (SEQ ID NO: 25) of the UniProtKB/Swiss-Prot database;

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (SEQ ID NO: 26) (residues 99-327) from UniProtKB/Swiss-Prot and CAC20457.1 (SEQ ID NO: 27) (residues 99-327) from GenBank.

In a specific embodiment, the Fc region is based on the amino acid sequence of a human IgG4 set out as P01861 (SEQ ID NO: 26) in UniProtKB/Swiss-Prot, residues 99-327, and has the amino acid sequence shown below and referenced herein as SEQ ID NO: 6:

(SEQ ID NO: 6)
ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

The Fc region can incorporate one or more alterations, usually not more than about 5 such alterations, including amino acid substitutions that affect certain Fc properties. In one specific and preferred embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F, and many others such as residue 409 alteration. Particularly useful are those alterations that enhance Fc properties while remaining silent with respect to conformation, e.g., retaining Fc receptor binding.

In a specific embodiment, and in the case where the Fc component is an IgG4 Fc, the Fc incorporates at least the S²²⁸P mutation, and has the amino acid sequence set out below and referenced herein as SEQ ID NO: 7:

(SEQ ID NO: 7)
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

The SIRPαFc fusion protein useful in the combination is one that inhibits the binding between human SIRPα and human CD47, thereby to inhibit or reduce transmission of the signal mediated via SIRPα-bound CD47, the fusion protein comprising a human SIRPα component and, fused therewith, an Fc component, wherein the SIRPα component comprises or consists of a single IgV domain of human SIRPα V2 and the Fc component is the constant region of a human IgG having effector function.

In one embodiment, the fusion protein comprises a SIRPα component comprising or consisting at least of residues 32-137 of the V2 form of wild type human SIRPα, i.e., SEQ ID NO: 1. In a preferred embodiment, the SIRPα component consists of residues 31-148 of the V2 form of human SIRPα, i.e., SEQ ID NO: 5. In another embodiment, the Fc component is the Fc component of the human IgG1 designated P01857 (SEQ ID NO: 15), and in a specific embodiment has the amino acid sequence that incorporates the lower hinge-CH2-CH3 region thereof i.e., SEQ ID NO: 2.

In a preferred embodiment, therefore, the present invention provides a SIRPαFc fusion protein, as an expressed single chain polypeptide and/or as a secreted dimeric fusion thereof, wherein the fusion protein incorporates a SIRPα component having SEQ ID NO: 1 and preferably SEQ ID NO: 5 and, fused therewith, an Fc region having effector function and having SEQ ID NO: 2. When the SIRPα component is SEQ ID NO: 1, this fusion protein comprises SEQ ID NO: 28, shown below:

(SEQ ID NO: 28)
EELQVIQPDKSVSVAAGESAILHCTVTSLIPVGPIQWFRGAGPARELIYN
QKEGHFPRVTTVSESTKRENMDFSISISNITPADAGTYYCVKFRKGSPDT
EFKSGAGTELSVRAKPSDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK*

When the SIRPα is SEQ ID NO: 5, this fusion protein comprises SEQ ID NO: 3, shown below:

(SEQ ID NO: 3)
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK

In alternative embodiments, the Fc component of the fusion protein is based on an IgG4, and preferably an IgG4 that incorporates the S²²⁸P mutation. In the case where the fusion protein incorporates the preferred SIRPα IgV domain of SEQ ID NO: 5, the resulting IgG4-based SIRPα-Fc protein comprises SEQ ID NO: 8, shown below:

(SEQ ID NO: 8)
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTESVRAKPSESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK

In preferred embodiment, the fusion protein comprises, as the SIRPα IgV domain of the fusion protein, a sequence that is SEQ ID NO: 5. The preferred SIRPαFc is SEQ ID NO: 3. In another embodiment, the SIRPαFc is SEQ ID NO: 8

The SIRPa sequence incorporated within SIRPαFc drug can be varied, as described in the literature. That is, useful substitutions within SIRPa include one or more of the following: L⁴V/I, V⁶I/L, A²¹V, V²⁷I/L, ¹³¹T/S/F, E⁴⁷V/L, K⁵³R, E⁵⁴Q, H⁵⁶P/R, S⁶⁶T/G, K⁶⁸R, V⁹²I, F⁹⁴V/L, V⁶³I, and/or F¹⁰³V, wherein amino acid position numbers are made with reference to SEQ ID NO: 5 herein; see also International Patent Publication No. WO2016/023040 (Alexo), incorporated herein by reference in its entirety.

In the SIRPαFc fusion protein, the SIRPα component and the Fc component are fused, either directly or indirectly, to provide a single chain polypeptide that is ultimately produced as a dimer in which the single chain polypeptides are coupled through intrachain disulfide bonds formed within the Fc region. The nature of the fusing region is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, ClaI, EcoRI, HindIII, PstI, SalI and XhoI site and the like.

The linker amino acids typically and desirably will provide some flexibility to allow the Fc and the SIRP components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G₄S sequence (Gly-Gly-Gly-Gly-Ser, SEQ ID NO: 11) which may repeat as (G₄S)ₙ where n is 1, 2, 3 or more, or is based on (Gly)n, (Ser)n, (Ser-Gly)n or (Gly-Ser)n and the like. In another embodiment, the linker is GTELSVRAKPS (SEQ ID NO: 4). This sequence constitutes SIRPα sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

The SIRPαFc fusion is useful to inhibit interaction between SIRPα and CD47, thereby to block signalling across this axis. Stimulation of SIRPα on macrophages by CD47 is known to inhibit macrophage-mediated phagocytosis by deactivating myosin-II and the contractile cytoskeletal activity involved in pulling a target into a macrophage. Activation of this cascade is therefore important for the survival of CD47+ disease cells, and blocking this pathway enables macrophages to eradicate the CD47+ disease cell population.

The term “CD47+” is used with reference to the phenotype of cells targeted for binding by the SIRPαFc drug. Cells that are CD47+ can be identified by flow cytometry using CD47 antibody as the affinity ligand. CD47 antibodies that are labeled appropriately are available commercially for this use (for example, clone B6H12 is available from Santa Cruz Biotechnology). The cells examined for CD47 phenotype can include standard tumour biopsy samples including particularly blood samples taken from the subject suspected of harbouring endogenous CD47+ cancer cells. CD47 disease cells of particular interest as targets for therapy with the present fusion proteins are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

The present drug combination comprises both a SIRPαFc drug that is preferably SEQ ID NO: 3, and a macrophage stimulating agent. These macrophage stimulating agents include a wide variety of agents that stimulate macrophage activity and affect macrophage polarity. In embodiments, the macrophage stimulating agent is a TLR agonist, a growth factor, or a chemokine.

It is well known that macrophages exist as different types, i.e., M0, M1 and M2, and that the M2 type has four different subtypes referred to as M2a, M2b, M2c and M2d. M1 and M2 macrophages have distinct chemokine and chemokine receptor profiles, with M1 secreting the Th1 cell attracting chemokines CXCL9 and CXCL10 and M2 secreting CCL17, CCL22 and CCL24. It has recently been demonstrated, in vitro, that macrophages are capable of complete repolarization from M2 to M1, and can reverse their polarization depending on their environment. The change in polarization is rapid and involves rewiring of signaling networks at both the transcriptional and translational levels.

The M1 phenotype results from activation by intracellular pathogens, bacterial cell wall components, lipoproteins, and cytokines such as interferon gamma (IFNg or IFNγ) and tumor necrosis factor alpha (TNFα). The M1 macrophages are characterized by inflammatory cytokine secretion and production of nitric oxide (NO), resulting in inflammation. In general M1 macrophages are associated with good prognosis in cancer settings. The M2 activation is induced by fungal cells, parasites, immune complexes, complements, apoptotic cells, interleukin-4 (IL-4), IL-13, IL-10, tumor growth factor beta (TGFβ). The M2 macrophages have been demonstrated to produce extracellular matrix (ECM) components, angiogenic and chemotactic factors, and IL-10. M2 macrophages can mitigate inflammatory response, and promote wound healing. They are widely termed in the current literature as anti-inflammatory, pro-resolving, wound healing, tissue repair, and trophic or regulatory macrophages and are considered benign opposites of the M1 activated macrophages.

Also, In accordance with the current framework for macrophage-activation nomenclature, instead of the traditional naming of the in vitro generated macrophages (M0, M1, M2a, M2b, M2c), the macrophage populations can be named in accordance with the agents that induce them, such as follows: M1 as M(IFN-γ), M1+LPS as M(IFNγ+LPS), M2a as M(IL-4), M2b as M(HAGG+IL-1β) and M2c as M(IL-10+TGFβ) subsets. Un-polarized M0 macrophages can be denoted as M(−).

As will be demonstrated in the examples provided herein, it is found that a SIRPαFc drug has an improved effect when the macrophage population is stimulated e.g., activated. Thus, in embodiments, a subject receiving a SIRPαFc drug is also treated with a macrophage stimulating agent that will cause macrophages to change phenotype, and to adopt an active state. Preferably, this is revealed as CD64 overexpression, or as adoption of an M1 or M2c type, or more simply as a result of treatment with a known macrophage stimulating agent.

Desirably, the treatment makes use of a macrophage stimulating agent that fosters or promotes the formation or accumulation of macrophages expressing CD64 (FcyRI). More particularly, the term “promotes formation” is intended to mean simply that an elevation in the number of certain macrophages is a result or consequence of administering that agent. Thus, an agent that promotes formation of CD64+ macrophages or fosters CD64+ macrophages is an agent that causes an increase in the number and prevalence of CD64+ macrophages, relative to CD16+ or CD32+ macrophages.

CD64, known also as FcyRI, functions on various types of immune cells as a receptor that binds to the Fc region of any protein, and particularly of antibodies. It is responsible for clearance of these proteins. In its human form, CD64 is a 75 kDa type I transmembrane protein having a known amino acid sequence. It is the high affinity receptor for IgG and is involved in phagocytosis, antibody-dependent cell-mediated cytotoxicity, and cytokine production. Monocytes and macrophages express CD64 constitutively. Mature granulocytes and lymphocytes are negative, but treatment of polymorphonuclear leukocytes with cytokines like IFNγ and G-CSF can induce CD64 expression on these cells as well.

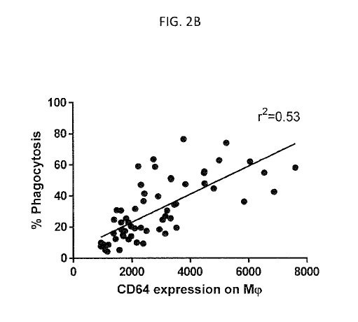

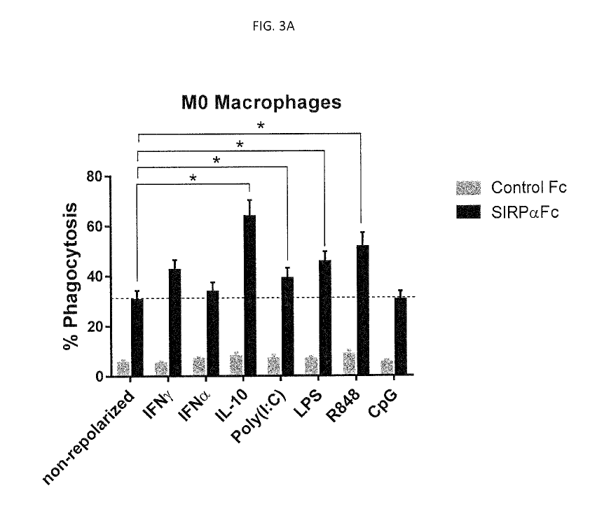

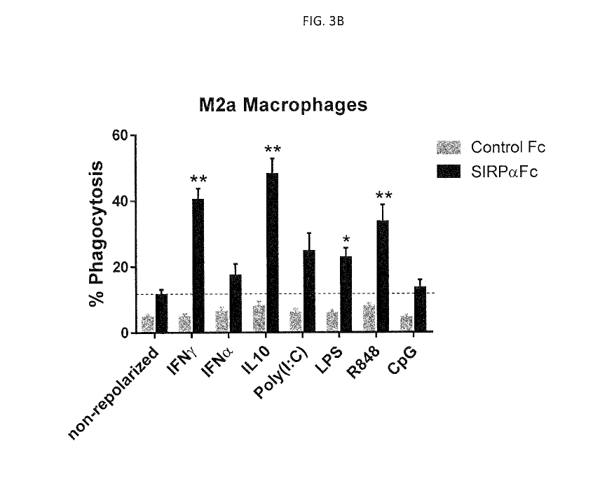

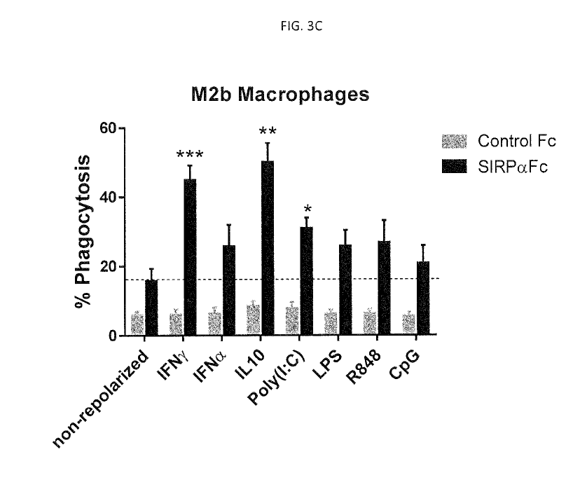

Thus, in embodiments, the macrophage stimulating agent is one that fosters CD64+ macrophages. The presence of these macrophages enhances the activity of the SIRPαFc drug. For example, FIG. 3 reveals some macrophage stimulating agents that cause CD64 to elevate, relative to other Fcy receptors CD16 and CD32, and relative to controls (compare the histogram shifts to the non-polarized control). This is most striking with IFN-alpha and IL-10. These can be used alone or in combination, together with SIRPαFc. Still other agents that foster CD64 macrophages can be identified using the experimental approach described herein

Other macrophage stimulating agents are very well known in the art. These are agents that cause macrophages to become active, and responsive to an insult. Agents with this property include numerous different components of microbial pathogens. These are recognized by different macrophage receptors referred to as toll-like receptors (TLRs).

In embodiments, the SIRPaFc drug is used in anti-cancer combination with at least one agent selected from:
1) an interferon selected from interferon-gamma (IFN-γ, Actimmune®) and an interferon-alpha such as interferon-alpha 2a (IFNα-2a, Pegasys®);
2) lipopolysaccharide (LPS), alone or together with an agent under 1) above;
3) an interleukin such as interleukin 1β, interleukin 4, and interleukin 10,
4) a protein such as colony stimulating factors such as M-CSF and GM-CSF, transforming growth factor beta (TGFβ), tumour necrosis factor alpha (TNFα), and heat aggregated human gamma globulin (HAGG),
5) a toll-like receptor (TLR) ligand, such as a TLR3, TLR4, TLR7 or TLR8 ligand, and mixtures of any two or more thereof.

In embodiments the macrophage stimulating agent used in combination with a SIRPαFc drug is a TLR agonist, i.e., an agent that binds and stimulates one of these receptors. As noted, toll-like receptors (TLRs) are pattern recognition receptors that macrophages (and other cells) use to recognize microbial structures (TLR3 recognizes double-stranded RNA, TLR4 recognizes LPS, TLR 7/8 recognize single-stranded RNA, TLR9 recognizes unmethylated CpG motifs). In general, the triggering of TLRs results in macrophage “activation” i.e., an increase in cytokine production, among other compounds. TLR stimulation also promotes polarization to the M1 macrophage phenotype.

Thus, in one embodiment, the present method utilizes the macrophage activating and polarizing effects of TLR agonists. These include agonists of any one or more of the TLRs including TLR-1, TLR-2, TLR-3, TLR-4, TLR-5, TLR-6, TLR-7, TLR-8 and/or TLR-9.

For TLR1, the agent can include bacterial and mycobacterial triacylated lipopeptides, including synthetic ligands such as tripalmitoyl-S-glyceryl-cysteine (Pam3Cys).

For TLR2, useful ligands include Gram positive bacterial peptidoglycan, bacterial lipoprotein, lipotechoic acid, certain LPS, GPI-anchor proteins from Trypanosoma cruzi, hemagglutinin (MV), phspholipomannan (Candida) and LAM (Mycobacteria) and Neisserial porins. Also know are synthetic ligands including complete Freund's adjuvant (CFA), macrophage activating lipopeptide 2 (MALP2), Hib-OMPC, S-(2,3-bispalmitoyloxypropyl)CGDPKHPKSF (FSL-1) (SEQ ID NO: 12), and dipalmitoyl-S-glyceryl-cysteine (Pam2Cys).

TLR3 is a nucleotide sensing TLR that binds the double stranded RNA produced by most viruses at some point during their replication. These viruses include West Nile virus, and double stranded RNA viruses such as RSV and MCMV. Synthetic ligands useful in the present method include polyinosine-polycytidylic acid (poly I:C), and polyadenylic-polyuridylic acid (poly A:U). In a preferred embodiment, the TLR agonist is poly(I:C), a long synthetic analog of dsRNA. It can be composed of a stand of poly (I) annealed to a strand of poly (C), and has an average size of 1.5-8 kb.

With TLR4, useful pathogen-borne ligands include LPS (Gram-negative bacteria); F-protein (RSV); Mannan (Candida); Glycoinositolphospholipids (Trypanosoma); Envelope proteins (RSV and MMTV). There are also endogenous ligands that bind TLR4, and these include Hsp60, Hsp70, fibronectin domain A, as well as hyaluronan, surfactant protein A and high mobility group 1 protein (HMGB-1). Synthetic TLR4 ligands are also known and include α-1 acid glycoprotein (AGP), monophosphoryl lipid A (MPLA), the lipid A mimetic designated RC-529, murine β defensin-2 (MDF2β) and CFA.

As for TLR5, bacterial flagellin serves as both a pathogenic and synthetic ligand useful in the present method.

With TLR6, useful pathogen-associated ligands are phenol-soluble modulin from Staph. Epidermidis, zymosan (Saccharomyces), LTA (Streptococcus) and diacylated polypeptides from Mycoplasma. Endogenous ligands are unknown but useful synthetic ligands include MALP-2, Pam2Cys and FSL-1.

For TLR7, useful synthetic ligands include guanosine analogs, Loxoribine, Resiquimod, R848, Aldara®, imidazoquinolines, and Imiquimod, whereas endogenous ligands are human RNA and pathogenic ligands are viral single stranded RNA particularly from Influenza, VSV, HIV and HCV.

For TLR8, single stranded RNA from RNA virus is the pathogen-derived ligand, whereas the endogenous ligand is human RNA and, similar to TLR7, the useful synthetic ligands include imidazoquinolines, Loxoribine, ss-poly-U, and 3M-012.

For TLR9, the pathogen-derived ligands include double stranded DNA viruses (HSV, MCMV), hemozoin from Plasmodium, and Unmethylated CpG DNA from bacteria and viruses. Endogenous ligands include human DNA/chromatin, and LL37-DNA. Useful synthetic ligands include CpG-based oligonucleotides.

Thus in embodiments, there is provided a method for depleting CD47+ disease cells by treating a subject in need thereof with a drug combination comprising a SIRPαFc drug and a TLR agonist effective to activate endogenous macrophages. In specific embodiments the TLR agonist is a physically tolerable agent selected from any of the TLR agonists just described. i.e., bacterial and mycobacterial triacylated lipopeptides, Pam3Cys, Gram positive bacterial peptidoglycan, bacterial lipoprotein, lipotechoic acid, LPS, GPI-anchor proteins from Trypanosoma cruzi, hemagglutinin (MV), phspholipomannan (Candida) and LAM (Mycobacteria), Neisserial porins, complete Freund's adjuvant (CFA), macrophage activating lipopeptide 2 (MALP2), Hib-OMPC, S-(2,3-bispalmitoyloxypropyl)CGDPKHPKSF (FSL-1), dipalmitoyl-S-glyceryl-cysteine, single stranded RNA viruses, double stranded RNA viruses such as RSV and MCMV poly I:C and poly A:U LPS (Gram-negative bacteria); F-protein (RSV); Mannan (Candida); Glycoinositolphospholipids (Trypanosoma); Envelope proteins (RSV and MMTV). Hsp60, Hsp70, fibronectin domain A, hyaluronan, surfactant protein A, high mobility group 1 protein (HMGB-1), α-1 acid glycoprotein (AGP), monophosphoryl lipid A (MPLA), the lipid A mimetic designated RC-529, murine β defensin-2 (MDF2β), bacterial flagellin, phenol-soluble modulin from Staph. Epidermidis, zymosan (Saccharomyces), diacylated polypeptides from Mycoplasma, MALP-2, Pam2Cys and FSL-1, guanosine analogs, Loxoribine, Resiquimod®, R848, Aldara®, imidazoquinolines, and Imiquimod, human RNA, viral single-stranded RNA, single stranded-poly-U, 3M-012, double stranded DNA virus, hemozoin from Plasmodium, unmethylated CpG DNA, human DNA/chromatin, LL37-DNA and CpG-based oligonucleotides. In other embodiments, the agonist is not a CpG-based oligonucleotide.

In particular embodiments, the preferred TLR agonist is an agonist at one of TLR3, TLR4, TLR7 and TLR8. In preferred embodiments, the TLR agonist is selected from the group consisting of lipopolysaccharide (LPS), R848 known also as Resiquimod®, and poly(I:C).

In some embodiments, the macrophage stimulating agent is not a TLR agonist.

The macrophage stimulating agent can also be any agent that drives macrophages in vitro to polarize or re-polarize into a desired macrophage type. This can be achieved using one of the TLR stimulating agents just described. In the alternative or in addition, the stimulating agent can be a cytokine or a growth factor such as macrophage colony stimulating factor (M-CSF) and granulocyte macrophage colony stimulating factor (GM-CSF), as well as transforming growth factor beta (TGFβ).

In one embodiment, the agent is an interferon. The interferon can be an interferon gamma (IFN-γ) such as particularly IFNγ-1b (Actimmune®) or an interferon alpha, such as interferon alpha-2a (IFNα-2a), or one of IFN-α1, IFN-α8, IFN-α10, IFN-α14 and IFN-α21.

In another embodiment, the agent is an interleukin. The interleukin can be interleukin-10 (IL-10), interleukin-4 (IL-4), or interleukin 1β.

Still other agents are useful provided they are able to activate macrophages in vitro toward a phenotype that is M1 or M2c. One such agent is heat aggregated human gamma globulin (HAGG).

Other macrophage stimulating agents useful herein are those that foster accumulation of CD64+ macrophages, i.e., cause an increase in the prevalence of CD64+ macrophages. These macrophages are shown to enhance activity of the SIRPαFc drug and particularly the SIRPa-based drug comprising SEQ ID NO: 3. Particularly useful macrophage stimulating agents that also foster CD64+ macrophages are interferon gamma 1b (Actimmune®) and interleukin-10. An assay useful to identify this type of macrophage stimulating agent is exemplified herein.

Macrophage stimulating agents can be used in combinations that, together with the SIRPαFc drug, can be IFN-γ and LPS; IL-10 and TGF-β; as well as HAGG and TGFβ.

In general, the SIRPαFc drug can also be used together with macrophage colony stimulating factor (M-CSF), or granulocyte macrophage colony stimulating factor (GM-CSF) in order to stimulate and activate the endogenous macrophages.

Each drug included in the combination can be formulated separately for use in combination. The drugs are said to be used “in combination” when the effect of one drug is used to augment the effect of the other, in a recipient of both drugs. Desirably, the treatment method entails administration of the macrophage stimulating agent first, followed by administration of the SIRPaFc drug at a time when the macrophages are stimulated by that agent.

In this approach, each drug is provided in a unit dosage form comprising a pharmaceutically acceptable carrier, and in a therapeutically effective amount. As used herein, “pharmaceutically acceptable carrier” means any and all solvents, dispersion media, coatings, antibacterial and antifungal agents, isotonic and absorption delaying agents, and the like that are physiologically compatible and useful in the art of protein/antibody formulation. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. The SIRPαFc fusion and the macrophage stimulating agent are formulated using practises standard in the art of formulating therapeutics. Solutions that are suitable for intravenous administration, such as by injection or infusion, are particularly useful.

Sterile injectable solutions can be prepared by incorporating the active compound in the required amount in an appropriate solvent with one or a combination of ingredients noted above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation are vacuum drying and freeze-drying (lyophilization) that yield a powder of the active ingredient plus any additional desired ingredient from a previously sterile-filtered solution thereof.

As used herein, “effective amount” refers to an amount effective, at dosages and for a particular period of time necessary, to achieve the desired therapeutic result. A therapeutically effective amount of each drug in the combination may vary according to factors such as the disease state, age, sex, and weight of the individual, and the ability of the drug to elicit a desired response in the recipient. A therapeutically effective amount is also one in which any toxic or detrimental effects of the pharmacological agent are outweighed by the therapeutically beneficial effects.

Effective amounts of each drug in the present combination will result in a number of depleted cancer cells that exceeds the number expected from the administration of either drug alone or independently.

The amount of active ingredient that can be combined with a carrier material to produce a unit dosage form will vary depending upon the subject being treated, and the particular mode of administration. The amount of active ingredient required to produce a single, unit dosage form will generally be that amount of the composition that produces a therapeutic effect. Generally, out of one hundred percent, this amount will range from about 0.01 percent to about ninety-nine percent of active ingredient, preferably from about 0.1 percent to about 70 percent, e.g., from about 1 percent to about 30 percent of active ingredient in combination with a pharmaceutically acceptable carrier.

For some of the macrophage stimulating agents, the amount useful in the present combination can be a dose that is already approved for human use. For instance, interferon gamma can be dosed using regimen similar to that required for treatment of chronic granulomatous disease or osteopetrosis applications. For interleukin 1β, one can use the dosing approved already for treatment of recurrent melanoma. When the macrophage stimulating agent is an already approved drug, one should consider dosing at a level lower than the approved dose, since the end-point of administration is for macrophage stimulation, and not for treating disease per se.

The SIRPαFc drug and the macrophage stimulating agent can be administered sequentially or, essentially at the same time. That is, the macrophage agent can be given before or after administration of the drug. It is desirable that the macrophage stimulating agent is administered first, so that the macrophages are stimulated when the SIRPαFc drug is administered.

Each drug in the combination can be administered separately, via one or more independently selected routes of administration using one or more of a variety of methods known in the art. As will be appreciated by the skilled artisan, the route and/or mode of administration will vary depending upon the desired results. Preferred routes of administration for proteins in the invention combination include intravenous, intramuscular, intradermal, intraperitoneal, subcutaneous, spinal or other parenteral routes for administration, for example by injection or infusion. The phrase “parenteral administration” that include injection such as intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In one embodiment, the SIRPaFc drug is administered intratumourally.

Alternatively, the drugs in the combination can be administered via a non-parenteral route, such as a by instillation or by a topical, epidermal or mucosal route of administration, for example, intranasally, orally, vaginally, rectally or sublingually.

Dosing regimens are adjusted to provide the optimum desired response (e.g., a therapeutic response). For example, a single bolus of each drug may be administered, or several divided doses may be administered over time or the dose may be proportionally reduced or increased as indicated by the therapeutic situation. It is especially advantageous to formulate parenteral compositions in unit dosage form for ease of administration and uniformity of dosage. “Unit dosage form” as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

The drugs can be formulated in combination, so that the combination can be introduced to the recipient in one administration, e.g., one injection or one infusion bag.

For administration of the drug combination, the dose for each drug will be within the range from about 0.0001 to 100 mg/kg, and more usually 0.01 to 5 mg/kg, of the host body weight. For example dosages can be 0.3 mg/kg body weight, 1 mg/kb body weight, 3 mg/kg body weight, 5 mg/kg body weight or 10 mg/kg body weight or within the range of 1-10 mg/kg. In unit dosage form, the SIRPαFc drug will comprise from 1-500 mgs of drug, such as 1, 2, 3, 4 5, 10 25, 50, 100, 200, 250, and 500 mgs/dose. The two drugs can be administered in roughly equimolar amounts (+/10%). An exemplary treatment regimen entails administration once per week, once every two weeks, once every three weeks, once every four weeks, once a month, once every 3 months or once every three to 6 months. Preferred dosage regimens for the drug combination of the invention include 1 mg/kg body weight or 3 mg/kg body weight via intravenous administration, with the drugs each being given simultaneously using one of the following dosing schedules; (i) every four weeks for six dosages, then every three months; (ii) every three weeks; (iii) 3 mg/kg body weight once followed by 1 mg/kg body weight every three weeks. In some methods, dosage is adjusted to achieve a plasma fusion protein concentration of about 1-1000 ug/ml and in some methods about 25-300 ug/ml.

The SIRPaFc drug displays negligible binding to red blood cells. There is accordingly no need to account for an RBC “sink” when dosing with drug combinations in which other SIRPαFc drugs are used. Relative to other SIRPαFc drugs that are bound by RBCs, it is estimated that the present SIRPaFc fusion can be effective at doses that are less than half the doses required for drugs that become RBC-bound, such as CD47 antibodies. Moreover, the SIRPα-Fc fusion protein is a dedicated antagonist of the SIRPα-mediated signal, as it displays negligible CD47 agonism when binding thereto. There is accordingly no need, when establishing medically useful unit dosing regimens, to account for any stimulation induced by the drug.

Each drug in the combination can also be administered as a sustained release formulation, in which case less frequent administration is required. Dosage and frequency vary depending on the half-life of the fusion protein in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient show partial or complete amelioration of symptoms of disease. Thereafter, the patient can be treated using a prophylactic regimen.

The drug combination is useful to “treat” a variety of CD47+ disease cells. Treatment can result in depletion of the targeted disease cells, i.e., in a reduction in the number of those cells as revealed for instance by a reduction in tumour size or distribution and, more directly, in a reduced number of circulating or solid tumour cells. The term “CD47+” is used with reference to the phenotype of cells targeted for binding by the present polypeptides. Cells that are CD47+ can be identified by flow cytometry using CD47 antibody as the affinity ligand. CD47 antibodies that are labeled appropriately are available commercially for this use (for example, clone B6H12 is available from Santa Cruz Biotechnology). The cells examined for CD47 phenotype can include standard tumour biopsy samples including particularly blood samples taken from the subject suspected of harbouring endogenous CD47+ cancer cells. CD47 disease cells of particular interest as targets for therapy are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

Cells that over-produce CD47 include particularly CD47+ cancer cells, including liquid and solid tumours. Solid tumours can be treated with the present drug combination, to reduce the size, number or growth rate thereof and to control growth of cancer stem cells. Such solid tumours include CD47+ tumours in bladder, brain, breast, lung, colon, ovary, prostate, liver and other tissues as well. In one embodiment, the drug combination is used to inhibit the growth or proliferation of hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma.

In some embodiments, the hematological cancer treated with the drug combination is a CD47+ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with the SIRPαFc protein is a CD47+ lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma as well as leimyosarcoma.

In one embodiment, the cancer is mycosis fungoides.

The combination therapy, comprising SIRPαFc and macrophage polarization, can also be exploited together with any other agent or modality useful in the treatment of the targeted indication, such as surgery as in adjuvant therapy, or with additional chemotherapy or radiation therapy as in neoadjuvant therapy.

### EXAMPLES

Trillium Therapeutics, Inc. provided pre-formulated SIRPαFc drugs and controls, as forms of soluble SIRPa designated (1) control Fc [human IgG1 region (hinge-CH2-CH3)], and (2) human SIRPaFc comprising the V region of human SIRPa variant 2 fused to a wild type human IgG1 Fc region (hinge CH2-CH3), as set out in SEQ ID NO: 3, which were stored at −80° C. until use.

### Example 1

Heparinized whole blood was obtained from normal healthy human donors (Biological Specialty Corporation) and informed consent was obtained from all donors. Peripheral blood mononuclear cells (PBMCs) were isolated over Ficoll-Paque Plus density gradient (GE Healthcare) and CD14+ monocytes were isolated from PBMCs by positive selection using CD14 antibody-coated MicroBead separation (Miltenyi Biotec). Monocytes were differentiated into macrophages by culturing for at least 10 days in X-Vivo-15 media (Lonza) supplemented with 20 ng/mL M-CSF (PeproTech). One day prior to the phagocytosis assay the monocyte-derived macrophages were either left untreated in M-CSF media (M0) or treated overnight with 20 ng/mL M-CSF and 300 ng/mL interferon-gamma (IFN-γ) (PeproTech) as M1, 50 ng/mL IFN-γ and 50 ng/mL LPS (MD Biosciences) as M1+LPS, 20 ng/mL IL-4 (PeproTech) as M2a, 20 ng/mL IL-1β (PeproTech) and 50 ug/mL heat aggregated human IgG (HAGG) as M2b or 20 ng/mL IL-10 (PeproTech) and 20 ng/mL TGF (PeproTech) as M2c. On the next day, macrophages were harvested using Enzyme-Free Cell Dissociation Buffer (ThermoFisher). Human B cell lymphoma cell line, Toledo, was labeled with Violet Proliferation Dye 450 (BD Biosciences) and these 6 subsets of macrophage in a round-bottom non-tissue culture treated 96-well plate at a 1:5 effector:target ratio. Macrophages and tumor cells were co-cultured for two hours at 3TC in 5% CO2 in the presence of SIRPaFc or control Fc protein. Cells were subsequently blocked with human Fc receptor binding inhibitor (ebioscience), followed by staining with Near-IR LIVE/DEAD Fixable Dead Cell Stain (Invitrogen), APC-conjugated anti-human CD14 (61D3, eBioscience) and PE-conjugated anti-human CD11b (ICRF44, eBioscience). They were washed and resuspended in Stabilizing Fixative (BD Biosciences). Cells were acquired on a FACSVerse flow cytometer, and data was analyzed using FlowJo software (Treestar Inc.). Macrophages were identified as live, single, CD14+CD11b+ cells. Doublets were excluded by SSC-W and SSC-H discrimination. % Phagocytosis was assessed as the % of macrophages that were VPD450+. Statistical significance was calculated by unpaired t-test vs isotype control using GraphPad Prism software, where p***<0.001.

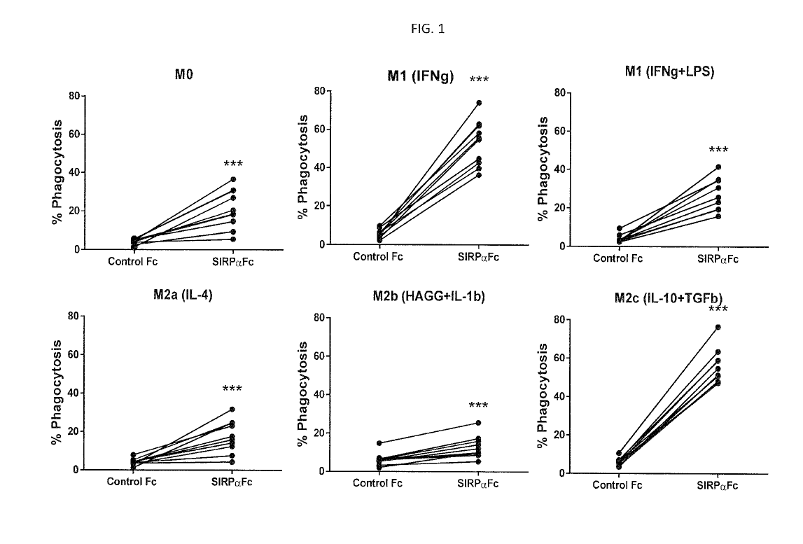

Results are shown in FIG. 1. As shown, blockade of CD47 on the tumor cells using 1 uM SIRPaFc increased phagocytosis of DLBCL Toledo cell line by all 6 macrophage subsets, with M1 (+/−LPS) and M2c MDMs being superior in SIRPαFc induced phagocytosis compared to M0, M2a and M2b subsets.

### Example 2

To further understand what drives the phagocytic capacity of polarized MDMs, we analyzed FcγR expression on the 6 distinctly polarized macrophages.

Macrophages were prepared as taught in Example 1. Then, macrophages were first blocked with human Fc receptor binding inhibitor (ebioscience), followed by staining with Near-IR LIVE/DEAD fixable dead cell stain (Invitrogen), FITC-conjugated anti-CD16 (Clone CB16), FITC-conjugated anti-CD32 (Clone FL18.26) and V450-conjugated anti-CD64 (Clone 10.1) in three independent cocktails. Cells were washed and resuspended in stabilizing fixative (BD Biosciences), and data was acquired on a FACSVerse flow cytometry machine, and data was analyzed using FlowJo software (Treestar Inc.). Macrophages were identified as live, single cells. Doublets were excluded by SSC-W and SSC-H discrimination. Percent of macrophage phagocytosis following 1 uM SIRPαFc treatment was analyzed as described in FIG. 1 and was plotted against the median fluorescent intensity of CD64.

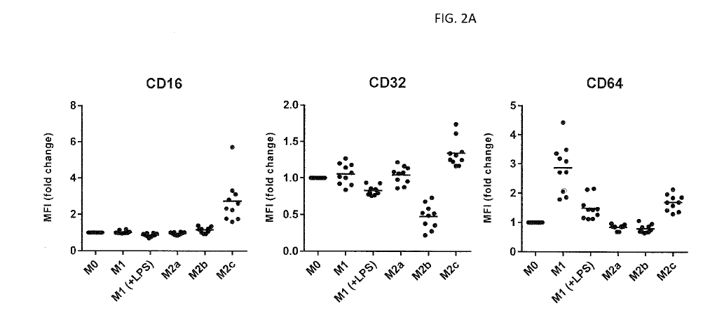

Results are summarized in FIG. 2A. It was found that M2c expresses highest level of CD16 and CD32, whereas M2b expresses lowest level of CD32. More interestingly, it was found that CD64 levels were highly variable, with M1 expressing highest level of CD64 followed by M1 (+LPS) and M2c, a pattern that correlated with the responsiveness to SIRPαFc. When % phagocytosis was plotted against CD64 expression across various macrophage subtypes across 10 independent donors, we observed a positive correlation between MDM expression of the high-affinity FcγRI (CD64) and phagocytic activity following SIRPαFc treatment, with r²=0.53 (FIG. 2B), whereas no significant correlation was found between % phagocytosis and CD32 or CD16 expression.

### Example 3

Heparinized whole blood was obtained from normal healthy human donors (Biological Specialty Corporation) and informed consent was obtained from all donors. Peripheral blood mononuclear cells (PBMCs) were isolated over Ficoll-Paque Plus density gradient (GE Healthcare) and CD14+ monocytes were isolated from PBMCs by positive selection using CD14 antibody-coated MicroBead separation (Miltenyi Biotec). Monocytes were differentiated into macrophages by culturing for at least 10 days in X-Vivo-15 media (Lonza) supplemented with 20 ng/mL M-CSF (PeproTech). Macrophages were washed and were polarized into M0, M2a, and M2b by culturing one day with 20 ng/mL M-CSF, 20 ng/mL IL-4 (PeproTech) or 50 ug/mL heat aggregated human gamma globulin (HAGG) and 20 ng/mL IL-1β (PeproTech), respectively. One day following polarization, polarization media was washed off, and cells were treated with 20 ng/mL IFN-γ (PeproTech), 1000 U/mL IFNα2a (PBL Assay Science), 20 ng/mL IL-10 (PeproTech), 10 ug/mL Poly (I:C) (InvivoGen), 1 ug/mL LPS (MDBiosciences), 1 ug/mL R848 (InvivoGen), or 10 ug/mL ODN2395 CpG (InvivoGen) overnight. On the next day, macrophages were harvested using enzyme-free cell dissociation buffer (ThermoFisher) for flow-based phagocytosis assay. Human B cell lymphoma cell line, Toledo, was labeled with Violet Proliferation Dye 450 (BD Biosciences) and added to repolarized macrophages in a round-bottom non-tissue culture treated 96-well plate at a 1:5 effector:target ratio. Macrophages and tumor cells were co-cultured for two hours at 37° C. in 5% CO2 in the presence of SIRPaFc or TTI-402, control Fc protein and subsequently blocked with human Fc receptor binding inhibitor (ebioscience) and stained with Near-IR LIVE/DEAD Fixable Dead Cell Stain (Invitrogen), APC-conjugated anti-human CD14 (61D3, eBioscience) and PE-conjugated anti-human CD11b (ICRF44, eBioscience), washed and resuspended in Stabilizing Fixative (BD Biosciences). Cells were acquired on a FACSVerse flow cytometer, and data was analyzed using FlowJo software (Treestar Inc.). Macrophages were identified as live, single, CD14+CD11b+ cells. Doublets were excluded by SSC-W and SSC-H discrimination. Phagocytosis was assessed as the % of macrophages that were VPD450+. Statistical significance was calculated by unpaired t-test vs isotype control using GraphPad Prism software.

Results are shown in FIGS. 3A-3C. (Statistical significance is shown with asterisks: *p≤0.05, **p≤0.01, ***p≤0.001.) It was demonstrated (FIG. 1) that M0, M2a and M2b MDMs exhibited lower phagocytic capabilities compared to M1 (+/−LPS) and M2c in response to SIRPaFc. Therefore, various cytokines and toll like receptor (TLR) agonists were used in attempt to re-polarize these 3 macrophage subsets into highly phagocytic MDM. It was found that the M0, M2a, and M2b were remarkably plastic in nature. Their responsiveness to SIRPaFc can be increased upon stimulation with repolarization using cytokines including IFN, IFNα, IL-10 and toll-like receptor agonists including Poly (I:C), LPS, R848, but not CpG.

### Example 4

Heparinized whole blood was obtained from normal healthy human donors (Biological Specialty Corporation) and informed consent was obtained from all donors. Peripheral blood mononuclear cells (PBMCs) were isolated over Ficoll-Paque Plus density gradient (GE Healthcare) and CD14+ monocytes were isolated from PBMCs by positive selection using CD14 antibody-coated MicroBead separation (Miltenyi Biotec). Monocytes were differentiated into macrophages by culturing for at least 10 days in X-Vivo-15 media (Lonza) supplemented with 20 ng/mL M-CSF (PeproTech). Macrophages were washed and were polarized into M0, M2a, and M2b by culturing one day with 20 ng/mL M-CSF, 20 ng/mL IL-4 (PeproTech) or 50 ug/mL heat aggregated human gamma globulin (HAGG) and 20 ng/mL IL-1β (PeproTech), respectively. One day following polarization, polarization media was washed off, and cells were treated with 20 ng/mL IFN-γ (Peprotech), 1000 U/mL IFNa2a (PBL Assay Science), 20 ng/mL IL-10 (PeproTech), 10 ug/mL Poly (I:C) (InvivoGen), 1 ug/mL LPS (MDBiosciences), 1 ug/mL R848 (InvivoGen), or 10 ug/mL ODN2395 CpG (InvivoGen) overnight. On the next day, macrophages were harvested using enzyme-free cell dissociation buffer (ThermoFisher) for analysis of CD16, CD32 and CD64 expression. Macrophages were blocked with human Fc receptor binding inhibitor (ebioscience) and were stained with Near-IR LIVE/DEAD fixable dead cell stain (Invitrogen), FITC-conjugated anti-CD16 (Clone CB16), FITC-conjugated anti-CD32 (Clone FL18.26) and V450-conjugated anti-CD64 (Clone 10.1) in three independent cocktails. Cells were washed and resuspended in stabilizing fixative (BD Biosciences), and data was acquired on a FACSVerse flow cytometry machine, and data was analyzed using FlowJo software (Treestar Inc.). Macrophages were identified as live, single cells. Doublets were excluded by SSC-W and SSC-H discrimination.

Representative results are shown in FIG. 4. It was demonstrated (FIG. 1) that M0, M2a and M2b MDMs exhibited slightly lower phagocytic capabilities compared to M1 (+/−LPS) and M2c in response to SIRPαFc. Therefore, various cytokines and toll like receptor (TLR) agonists were used to re-polarize these 3 macrophage subsets into highly phagocytic MDM. It was found that the M0, M2a, and M2b were remarkably plastic in nature. Their expression of CD64 can be increased upon overnight stimulation and repolarization with cytokines including IFNα, IFNγ, IL-10 and toll-like receptor agonists including Poly (I:C), but not LPS, R848 and CpG.

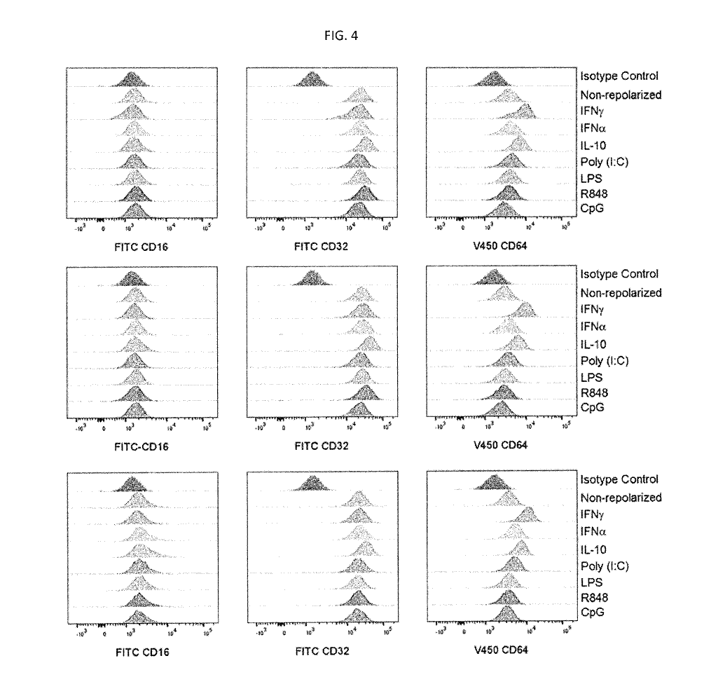

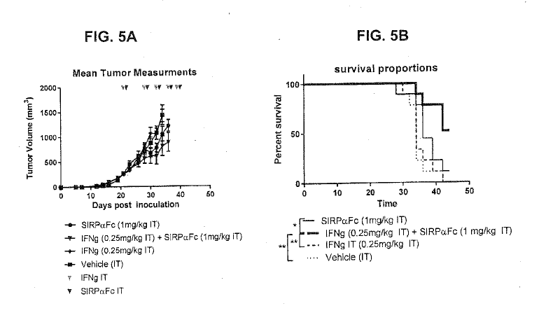

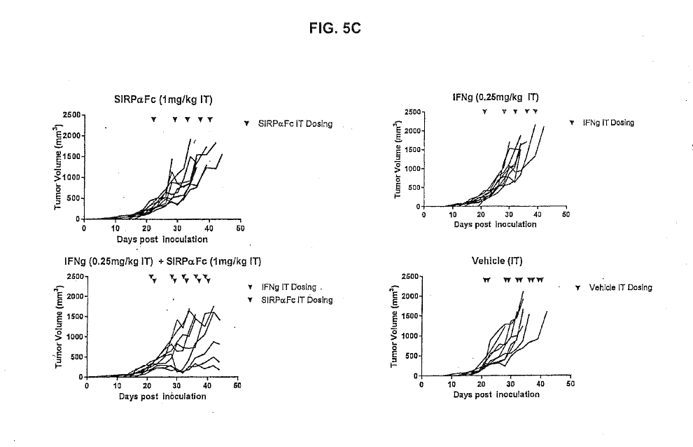

As shown in FIGS. 5A-5C, the impact of SIRPaFc (SEQ ID NO: 3) and IFN-gamma were examined as follows: 1×10⁷ Toledo cells in Matrigel were implanted subcutaneously into the right flank of SHrN NOD.SCID mice (n=9 mice per group) on day 0. Mice were randomized when the mean tumor size was approximately 260 mm3 and received intratumoral (IT) injections of SIRPaFc 1 mg/kg or IFNg 0.25 mg/kg or the combination of the two or vehicle. In the combination treatment, IFNg was dosed a day prior to SIRPaFc. IFNg and/or SIRPaFc or vehicle dosing was done on a weekly basis during the first two doses, and the frequency was increased to twice per week after the second dose. The study was terminated on day 44 post tumor inoculation (2 days after the all the vehicle treated mice reached the endpoint). In FIG. 5A, the mean tumor volume with standard mean deviation of each treatment group is shown. The curves terminated when more than 25% of the mice per group were sacrificed. The dosing schedule was indicated as inverted triangles. FIG. 5B shows survival of the tumor bearing mice. Statistical analysis of the survival curves was performed using log rank test, where indicated *. *p≤0.05, **p≤0.01. FIG. 5C shows individual tumor growth spider plot of each treatment group.

Although preferred embodiments of the invention have been described herein, it will be understood by those skilled in the art that variations may be made thereto without departing from the spirit of the invention or the scope of the appended claims. All documents disclosed herein are incorporated by reference.

1. A method for depleting CD47+ disease cells in a subject in need thereof, comprising administering, to the subject, a SIRPαFc drug and a macrophage stimulating agent.

2. (canceled)

3. The method according to claim 1, wherein the macrophage stimulating agent is a TLR agonist.

4. The method according to claim 3, wherein the TLR agonist is selected from lipopolysaccharide (LPS), Resiquimod, and poly(I:C).

5. (canceled)

6. The method according to claim 1, wherein the macrophage stimulating agent comprises at least one protein selected from macrophage colony stimulating factor (M-CSF), granulocyte macrophage colony stimulating factor (GM-CSF), heat aggregated gamma globulin (HAGG), tumour necrosis factor alpha (TNFα), and transforming growth factor beta (TGFβ).

7. The method according to claim 1, wherein the macrophage stimulating agent comprises an interferon selected from interferon gamma and interferon alpha.

8. The method according to claim 1, wherein the macrophage stimulating agent comprises an interleukin selected from IL-1β, IL-4, and IL-10, and mixtures thereof.

9. (canceled)

10. The method according to claim 1, wherein the SIRPαFc drug comprises an Fc based on IgG1.

11. The method according to claim 1, wherein the SIRPαFc drug comprises an Fc based on IgG4.

12. The method according to claim 10, wherein the SIRPαFc drug comprises the amino acid sequence of SEQ ID NO: 3.

13. The method according to claim 11, wherein the SIRPαFc drug comprises the amino acid sequence of SEQ ID NO: 8.

14. (canceled)

15. In combination in unit dosage form for depleting CD47+ disease cells in a subject in need thereof, a SIRPαFc drug effective for depleting CD47+ disease cells, and a macrophage stimulating agent effective for enhancing said depletion of CD47+ disease cells.

16. (canceled)

17. The method according to claim 1, wherein the CD47+ disease cells are CD47+ cancer cells.

18. The method according to claim 17, wherein the CD47+ cancer cells are CD47+ blood cancer cells.

19. The method according to claim 18, wherein the CD47+ cancer cells are cells of a cancer type selected from acute lymphocytic leukemia (ALL); acute myeloid leukemia (AML); chronic lymphocytic leukemia (CLL); chronic myelogenous leukemia (CML); myeloproliferative disorder/neoplasm (MPDS), mycosis fungoides; and myelodysplastic syndrome.

20. The method according to claim 19, wherein the cancer is a lymphoma selected from a Hodgkin's lymphoma, both indolent and aggressive non-Hodgkin's lymphoma, Burkitt's lymphoma, small cell follicular lymphoma and large cell follicular lymphoma.

21. The method according to claim 20, wherein the cancer is a myeloma selected from multiple myeloma (MM), giant cell myeloma, heavy-chain myeloma, and light chain or Bence-Jones myeloma.

22. The method according to claim 1, wherein the subject has a CD47+ cancer, and wherein the macrophage stimulating agent comprises interferon gamma 1 b or interferon alpha 2a.

23.-24. (canceled)

25. The combination in unit dosage form according to claim 15, wherein the SIRPαFc drug comprises SEQ ID NO: 3.

26. (canceled)

27. The combination in unit dosage form according to claim 15, wherein the SIRPαFc drug comprises SEQ ID NO: 8.

28.-29. (canceled)

30. A method of enhancing the anti-cancer effect of a SIRPαFc drug, the method comprising administering, to a subject receiving said SIRPαFc drug, a macrophage stimulating agent.
